(19) United States
(12) Patent Application Publication
(10) Pub. No.: US 2006/0073541 A1
(43) Pub. Date: Apr. 6, 2006

(54) Lipophilic dyes and their application for detection of myelin

(76) Inventor: Jason J. Kilgore, Junction City, OR (US)

Correspondence Address: KOREN ANDERSON;MOLECULAR PROBES, INC., 29851 WILLOW CREEK ROAD, EUGENE, OR, 97402-9132, US

(21) Appl. No.: 11/241,323

(22) Filed: Sep. 30, 2005

Related U.S. Application Data
(60) Provisional application No. 60/615,131, filed on Oct. 1, 2004.

(51) Int. Cl. G01N 33/48; C07D 213/26

(52) U.S. Cl. 435/40.5; 546/329

(57) ABSTRACT

Embodiments of the present invention provide a method for selectively detecting myelin in tissue samples using lipophilic dyes and kits for detecting myelin in a sample. The dyes of the present invention are represented by the general formula A-B-E wherein A is a nitrogen heterocycle, B is a bridge moiety and E is an electron pair accepting moiety that comprises either a carbonyl or nitrogen atom. In one embodiment these lipophilic dyes are a merocyanine dye, a cyanine dye, a styryl dye or a carbazolylvinyl dye.

### CROSS-REFERENCE TO RELATED APPLICATIONS

This application claims priority to U.S. Provisional Patent Application Ser. No. 60/615,131, filed Oct. 1, 2004, which disclosure is herein incorporated by reference.

### FIELD OF THE INVENTION

The invention relates to colored and fluorescent dyes, and to their use in staining myelin in samples. The invention has applications in the fields of cell biology, neurology, nutrition, immunology, and cancer biology.

### BACKGROUND OF THE INVENTION

The preparation and characterization of merocyanine dyes has been well documented. A large number of useful styryl merocyanine dyes (commonly referred to as RH dyes) have been previously prepared by Rina Hildesheim (Grinvald et al., BIOPHYS. J. 39, 301 (1982), Leslie Loew (Loew et al., J. ORG. CHEM. 49, 2546 (1984) and others, as useful probes for measuring electric potentials in cell membranes. Useful membrane potential measurements only occur in live cells and artificial liposomes, where the fluorescence intensity of a suitable dye as it is associated with the membrane changes as the membrane is subjected to an electrical gradient. In addition to the above membrane potential probes, an extensive variety of other merocyanine dyes have been described by Brooker et al. (J. AM. CHEM. SOC. 73, 5326 (1951)) primarily for use in the photographic industry, although Brooker et al. do not describe the fluorescence properties of the merocyanines.

The present invention describes the use of these and other lipophilic dyes to selectively detect myelin in tissue samples. Traditional methods for detection of myelin require use of antibodies, such as anti-myelin basic protein, or non-fluorescent (transmitted light) methods such as the Loyez method (Cook, H. C. 1974. Manual of Histological Demonstration Techniques. London: Butterworths. pp. 161-162.); Schmued's gold chloride technique (Schmued, L. C. 1990. A rapid, sensitive histochemical stain for myelin in frozen brain sections. J Histochem Cytochem. 38(5):717-20); Weil's Myelin Stain; Luxol Fast Blue (Sheehan, D. and Hrapchak, B. 1980. Theory and Practice of Histotechnology, 2^{nd} Ed. Battelle Press, Ohio. pp 262-264); Black Gold (Schmued, L. and Sklikker, W. Jr. 1999. Black-gold: a simple, high-resolution histochemical label for normal and pathological myelin in brain tissue sections. Brain Res. 837(1-2):289-97); Mulligan's Myelin Method; Sudan Black B (Stilwell, D. L. 1957. A sudan black B myelin stain for peripheral nerves. Stain Technol. 32(1):19-23; Gerrits, P. O. et al. 1992. Staining myelin and myelin-like degradation products in the spinal cords of chronic experimental allergic encephalomyelitis (Cr-EAE) rats using Sudan Black B staining of glycol methacrylate-embedded material, Journal of Neuroscience Methods 45: 99-105), all of which are time consuming, requiring multiple steps extending from one to three days. The present dyes, in contrast, require only a single 20-minute label step plus washes. These dyes can be used in combination with antibodies and other dyes, and with standard histochemical methods employed with cryosectioned material.

### SUMMARY OF THE INVENTION

Embodiments of the present invention provide a method for selectively detecting myelin in tissue samples using lipophilic dyes and kits for detecting myelin in a sample.

In an exemplary embodiment, the invention provides a method for the selective detection of myelin in a sample. The method comprises:
a) contacting the sample with a lipophilic fluorescent dye that selectively associates with myelin to prepare a labeling mixture;
b) incubating the labeling mixture for a sufficient amount of time for the dye to associate with the myelin to form an incubated sample;
c) illuminating the incubated sample with an appropriate wavelength to form an illuminated sample; and
d) observing the illuminated sample whereby the myelin is detected.

In one aspect the sample is a tissue section, typically a brain tissue section.

In one embodiment the method further comprises washing the sample to remove unbound dye before the illuminating step. In another embodiment the method further comprises contacting the sample with an additional detection reagent, before, during or after the sample has been contacted with the present lipophilic dye.

In one aspect the additional detection reagent is an antibody, a nucleic acid stain, an ion indicator, a cytoskeleton stain, an extracellular matrix stain or an organelle stain. In another aspect the organelle stain is selective for mitochondria, lysozymes, nucleus, golgi, or endoplastic reticulum (ER).

The dyes of the present invention are represented by the general formula A-B-E wherein A is a nitrogen heterocycle, B is a bridge moiety and E is an electron pair accepting moiety that comprises either a carbonyl or nitrogen atom. In one embodiment these lipophilic dyes are a merocyanine dye, a cyanine dye, a styryl dye or a carbazolylvinyl dye. Selected dye embodiments include dyes that are represented by Formula VI, VII and IX. A particular advantageous dye for detection of myelin is Compound 1, 2 or 4.

In another exemplary embodiment, the present invention provides kits for selective detection of myelin in a sample, wherein the kit comprises a present lipophilic dye and instructions for detecting myelin. In one aspect, the kit further comprises an additional detection reagent, buffer components and/or controls.

### BRIEF DESCRIPTION OF THE DRAWINGS

FIG. 1: Shows a black and white photograph of a tissue section containing myelin stained with A) Anti-Myelin Basic Protein, B) a chromogenic technique I) Schmued's Gold Chloride Technique, II) Loyez Technique, C) Compound 1 and D) Compound 2. See, Example 1.

FIG. 2: Shows comparison of myelin staining with anti-MBP and Compound 1. See Example 7.

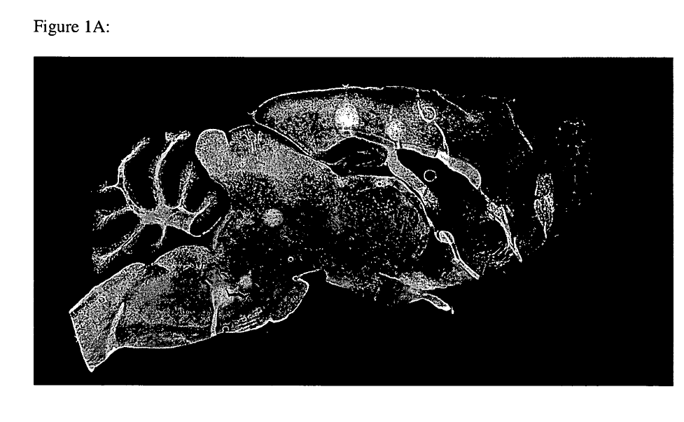

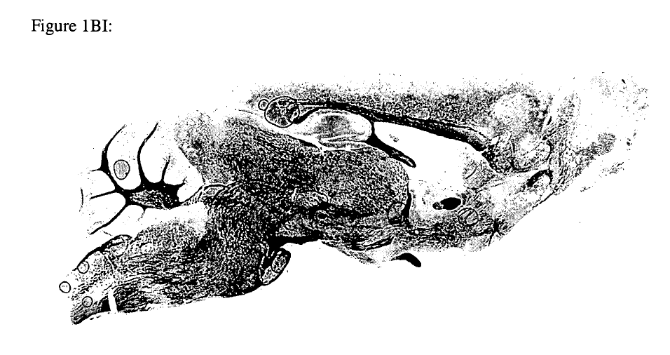

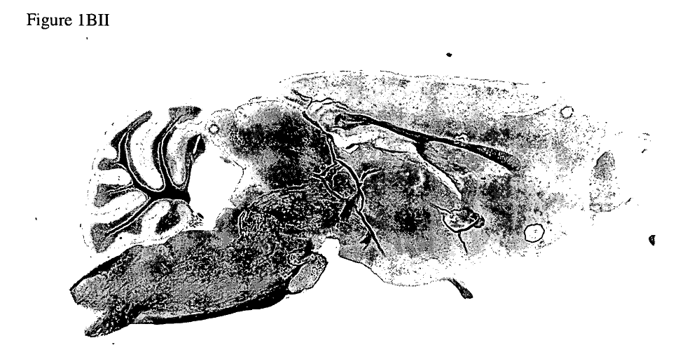

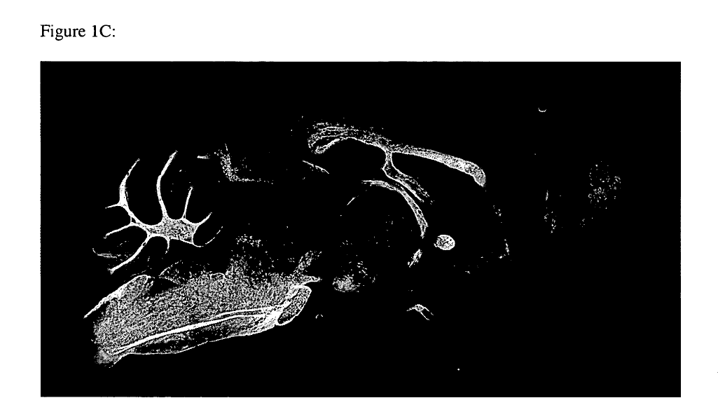

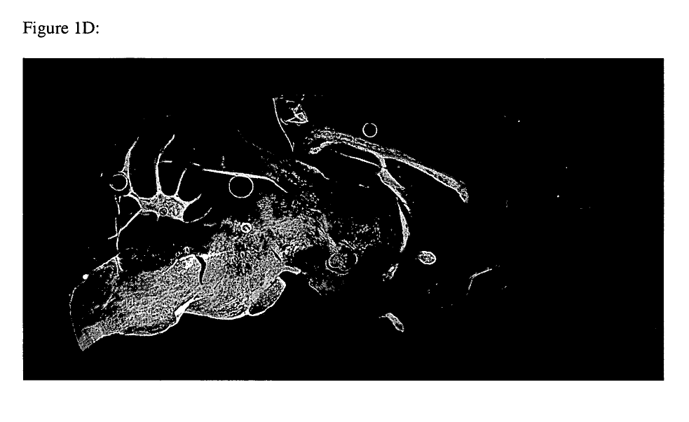

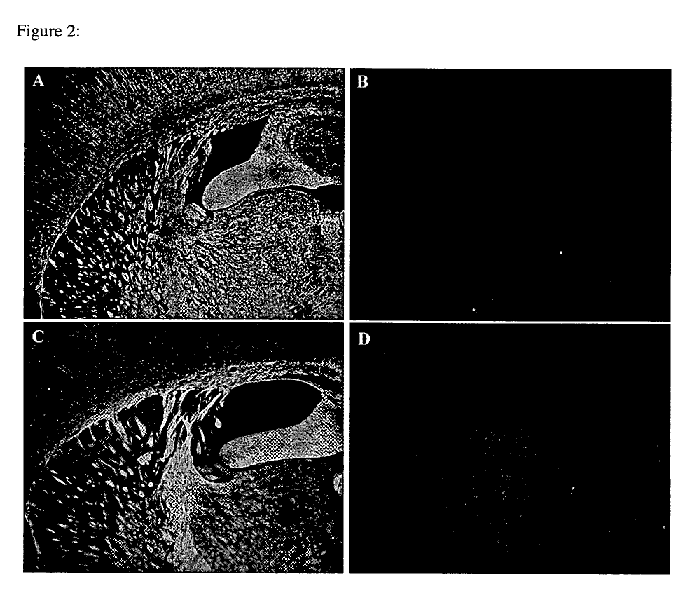

### DETAILED DESCRIPTION OF THE INVENTION

Introduction

The present invention describes lipophilic dyes, including cyanine and merocyanine dyes, and their application for staining myelin in brain tissue sections.

Definitions

Before describing the present invention in detail, it is to be understood that this invention is not limited to specific compositions or process steps, as such may vary. It must be noted that, as used in this specification and the appended claims, the singular form “a”, “an” and “the” includes plural referents unless the context clearly dictates otherwise. Thus, for example, reference to “a fluorescent dye” includes a plurality of dyes and reference to “a compound” includes a plurality of compounds and the like.

Unless defined otherwise, all technical and scientific terms used herein have the same meaning as commonly understood by one of ordinary skill in the art to which this invention is related. The following terms are defined for purposes of the invention as described herein.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.),VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

Although typically not shown for the sake of clarity, any overall positive or negative charges possessed by any of the compounds of the invention are balanced by a necessary counterion or counterions. Where the compound of the invention is positively charged, the counterion is typically selected from, but not limited to, chloride, bromide, iodide, sulfate, alkanesulfonate, arylsulfonate, phosphate, perchlorate, tetrafluoroborate, tetraarylborate, nitrate, hexafluorophosphate, and anions of aromatic or aliphatic carboxylic acids. Where the compound of the invention is negatively charged, the counterion is typically selected from, but not limited to, alkali metal ions, alkaline earth metal ions, transition metal ions, ammonium or substituted ammonium ions. Preferably, any necessary counterion is biologically compatible, is not toxic as used, and does not have a substantially deleterious effect on biomolecules. Counterions are readily changed by methods well known in the art, such as ion-exchange chromatography, or selective precipitation.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical. The “acyl radical” is the group derived from a carboxylic acid by removing the —OH moiety therefrom.

The term “alkyl,” by itself or as part of another substituent means, unless otherwise stated, a straight or branched chain, or cyclic hydrocarbon radical, or combination thereof, which may be fully saturated, mono- or polyunsaturated and can include divalent (“alkylene”) and multivalent radicals, having the number of carbon atoms designated (i.e. C ₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

Exemplary alkyl groups of use in the present invention contain between about one and about twenty-five carbon atoms (e.g. methyl, ethyl and the like). Straight, branched or cyclic hydrocarbon chains having eight or fewer carbon atoms will also be referred to herein as “lower alkyl”. In addition, the term “alkyl” as used herein further includes one or more substitutions at one or more carbon atoms of the hydrocarbon chain fragment.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain, or cyclic carbon-containing radical, or combinations thereof, consisting of the stated number of carbon atoms and at least one heteroatom selected from the group consisting of O, N, Si, P and S, and wherein the nitrogen, phosphorous and sulfur atoms are optionally oxidized, and the nitrogen heteroatom is optionally be quaternized, and the sulfur atoms are optionally trivalent with alkyl or heteroalkyl substituents. The heteroatom(s) O, N, P, S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means, unless otherwise stated, a polyunsaturated, aromatic moiety that can be a single ring or multiple rings (preferably from 1 to 4 rings), which are fused together or linked covalently. Specific examples of aryl substituents include, but are not limited to, substituted or unsubstituted derivatives of phenyl, biphenyl, o-, m-, or p-terphenyl, 1-naphthyl, 2-naphthyl, 1-, 2-, or 9-anthryl, 1-, 2-, 3-, 4-, or 9-phenanthrenyl and 1-, 2- or 4-pyrenyl. Preferred aryl substituents are phenyl, substituted phenyl, naphthyl or substituted naphthyl.

The term “heteroaryl” as used herein refers to an aryl group as defined above in which one or more carbon atoms have been replaced by a non-carbon atom, especially nitrogen, oxygen, or sulfur. For example, but not as a limitation, such groups include furyl, tetrahydrofuryl, pyrrolyl, pyrrolidinyl, thienyl, tetrahydrothienyl, oxazolyl, isoxazolyl, triazolyl, thiazolyl, isothiazolyl, pyrazolyl, pyrazolidinyl, oxadiazolyl, thiadiazolyl, imidazolyl, imidazolinyl, pyridyl, pyridaziyl, triazinyl, piperidinyl, morpholinyl, thiomorpholinyl, pyrazinyl, piperainyl, pyrimidinyl, naphthyridinyl, benzofuranyl, benzothienyl, indolyl, indolinyl, indolizinyl, indazolyl, quinolizinyl, qunolinyl, isoquinolinyl, cinnolinyl, phthalazinyl, quinazolinyl, quinoxalinyl, pteridinyl, quinuclidinyl, carbazolyl, acridinyl, phenazinyl, phenothizinyl, phenoxazinyl, purinyl, benzimidazolyl and benzthiazolyl and their aromatic ring-fused analogs. Many fluorophores are comprised of heteroaryl groups and include, without limitations, xanthenes, oxazines, benzazolium derivatives (including cyanines and carbocyanines), borapolyazaindacenes, benzofurans, indoles and quinazolones.

Where a ring substituent is a heteroaryl substituent, it is defined as a 5- or 6-membered heteroaromatic ring that is optionally fused to an additional six-membered aromatic ring(s), or is fused to one 5- or 6-membered heteroaromatic ring. The heteroaromatic rings contain at least 1 and as many as 3 heteroatoms that are selected from the group consisting of O, N or S in any combination. The heteroaryl substituent is bound by a single bond, and is optionally substituted as defined below.

Specific examples of heteroaryl moieties include, but are not limited to, substituted or unsubstituted derivatives of 2- or 3-furanyl; 2- or 3-thienyl; N-, 2- or 3-pyrrolyl; 2- or 3-benzofuranyl; 2- or 3-benzothienyl; N-, 2- or 3-indolyl; 2-, 3- or 4-pyridyl; 2-, 3- or 4-quinolyl; 1-, 3-, or 4-isoquinolyl; 2-, 4-, or 5-(1,3-oxazolyl); 2-benzoxazolyl; 2-, 4-, or 5-(1,3-thiazolyl); 2-benzothiazolyl; 3-, 4-, or 5-isoxazolyl; N-, 2-, or 4-imidazolyl; N-, or 2-benzimidazolyl; 1- or 2-naphthofuranyl; 1- or 2-naphthothienyl; N-, 2- or 3-benzindolyl; 2-, 3-, or 4-benzoquinolyl; 1-, 2-, 3-, or 4-acridinyl. Preferred heteroaryl substituents include substituted or unsubstituted 4-pyridyl, 2-thienyl, 2-pyrrolyl, 2-indolyl, 2-oxazolyl, 2-benzothiazolyl or 2-benzoxazolyl.

The above heterocyclic groups may further include one or more substituents at one or more carbon and/or non-carbon atoms of the heteroaryl group, e.g., alkyl; aryl; heterocycle; halogen; nitro; cyano; hydroxyl, alkoxyl or aryloxyl; thio or mercapto, alkyl- or arylthio; amino, alkyl-, aryl-, dialkyl-, diaryl-, or arylalkylamino; aminocarbonyl, alkylaminocarbonyl, arylaminocarbonyl, dialkylaminocarbonyl, diarylaminocarbonyl or arylalkylaminocarbonyl; carboxyl, or alkyl- or aryloxycarbonyl; aldehyde; aryl- or alkylcarbonyl; iminyl, or aryl- or alkyliminyl; sulfo; alkyl- or arylsulfonyl; hydroximinyl, or aryl- or alkoximinyl. In addition, two or more alkyl substituents may be combined to form fused heterocycle-alkyl ring systems. Substituents including heterocyclic groups (e.g., heteroaryloxy, and heteroaralkylthio) are defined by analogy to the above-described terms.

The term “heterocycloalkyl” as used herein refers to a heterocycle group that is joined to a parent structure by one or more alkyl groups as described above, e.g., 2-piperidylmethyl, and the like. The term “heterocycloalkyl” refers to a heteroaryl group that is joined to a parent structure by one or more alkyl groups as described above, e.g., 2-thienylmethyl, and the like.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present. In the schemes that follow, the symbol X represents “R” as described above.

The aryl and heteroaryl substituents described herein are unsubstituted or optionally and independently substituted by H, halogen, cyano, sulfonic acid, carboxylic acid, nitro, alkyl, perfluoroalkyl, alkoxy, alkylthio, amino, monoalkylamino, dialkylamino or alkylamido.

Two of the substituents on adjacent atoms of the aryl or heteroaryl ring may optionally be replaced with a substituent of the formula -T-C(O)—CRR′)_{q}-U- wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)S—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), phosphorus (P) and silicon (Si).

The term “amino” or “amine group” refers to the group —NR′R″ (or NRR′R″) where R, R′ and R″ are independently selected from the group consisting of hydrogen, alkyl, substituted alkyl, aryl, substituted aryl, aryl alkyl, substituted aryl alkyl, heteroaryl, and substituted heteroaryl.

A substituted amine being an amine group wherein R′ or R″ is other than hydrogen. In a primary amino group, both R′ and R″ are hydrogen, whereas in a secondary amino group, either, but not both, R′ or R″ is hydrogen. In addition, the terms “amine” and “amino” can include protonated and quaternized versions of nitrogen, comprising the group —NRR′R″ and its biologically compatible anionic counterions.

The term “aqueous solution” as used herein refers to a solution that is predominantly water and retains the solution characteristics of water. Where the aqueous solution contains solvents in addition to water, water is typically the predominant solvent.

The term “buffer” as used herein refers to a system that acts to minimize the change in acidity or basicity of the solution against addition or depletion of chemical substances.

The term “carbonyl” as used herein refers to the functional group —(C═O)—. However, it will be appreciated that this group may be replaced with other well-known groups that have similar electronic and/or steric character, such as thiocarbonyl (—(C═S)—); sulfinyl (—S(O)—); sulfonyl (—SO₂)—), phosphonyl (—PO₂—).

The term “carboxy” or “carboxyl” refers to the group —R′(COOR) where R′ is alkyl, substituted alkyl, aryl, substituted aryl, arylalkyl, substituted arylalkyl, heteroaryl, or substituted heteroaryl. R is hydrogen, a salt or —CH₂OC(O)CH₃.

The term “detectable response” as used herein refers to a change in or an occurrence of, a signal that is directly or indirectly detectable either by observation or by instrumentation. Typically, the detectable response is an optical response resulting in a change in the wavelength distribution patterns or intensity of absorbance or fluorescence or a change in light scatter, fluorescence lifetime, fluorescence polarization, or a combination of the above parameters. Alternatively, the detectable response is an occurrence of a signal wherein the fluorophore is inherently fluorescent and does not produce a change in signal when in contact with the sample. Alternatively, the detectable response is the result of a signal, such as color, fluorescence, radioactivity or another physical property of the fluorophore becoming spatially localized in a subset of a sample such as in a gel, on a blot, or an array, in a well of a micoplate, in a microfluidic chamber, or on a microparticle as the result of non-covalent association within the sample.

The term “directly detectable” as used herein refers to the presence of a detectable label or the signal generated from a detectable label that is immediately detectable by observation, instrumentation, or film without requiring chemical modifications or additional substances. For example, a fluorophore produces a directly detectable response.

The term “kit” as used herein refers to a packaged set of related components, typically one or more compounds or compositions.

The term “salt thereof,” as used herein includes salts of the agents of the invention and their conjugates, which are preferably prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of base addition salts include sodium, potassium, calcium, ammonium, organic amino, or magnesium, or a similar salt. When compounds of the present invention contain relatively basic functionalities, acid addition salts can be obtained by contacting the neutral form of such compounds with a sufficient amount of the desired acid, either neat or in a suitable inert solvent. Examples of addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The term “sample” as used herein refers to any material that may contain myelin. Typically the myelin is associated with nerves, such as found in brain tissue section. The sample may comprise a purified or semi-purified synthetic proteins and endogenous host cell proteins, tissue homogenate, bodily and other biological fluids, or synthesized proteins, all of which comprise a sample in the present invention. The sample may be in an aqueous or mostly aqueous solution, a viable cell culture or immobilized on a solid or semi solid surface such as a microscope slide, a polymer gel, a membrane, a microparticle, an optical fiber or on a microarray.

As used herein the term “sulfonic acid” means either —SO₃H, or a salt of sulfonic acid. Also as used herein the term “carboxylic acid” means either —COOH, or a salt of carboxylic acid. Appropriate salts of sulfonic and carboxylic acids include, among others, K⁺, Na⁺, Cs⁺, Li⁺, Ca²⁺, Mg²⁺, ammonium, alkylammonium or hydroxyalkylammonium salts, or pyridinium salts. Alternatively, the counterion of the sulfonic acid or carboxylic acid may form an inner salt with a positively charged atom on the dye itself, typically a quaternary nitrogen atom.

The Compounds

In general, for ease of understanding the present invention, the lipophilic dye compounds and corresponding substituents will first be described in detail, followed by the methods in which the compounds find uses, which is followed by exemplified methods of use.

The present methods selectively stain myelin in a sample, typically a tissue section that has been immobilized on a glass surface. A wide range of lipophilic dyes are envisioned by the present invention and there is no intended limitation of the lipophilic dye that can be used with the present methods, provided that the dye selectively associates with myelin.

Lipophilic dyes include, but are not limited to, cyanine dyes, merocyanine dyes, styryl dyes and carbazolylvinyl dyes. As used herein “lipophilic” means a dye that comprises a carbon chain that contains at least three carbons. These carbon chains are present in the form of an alkyl chain or a substituted alkyl chain and allow the dye to interact with hydrophobic moieties such as lipids, certain detergents such as sodium dodecyl sulfate (SDS) and hydrophobic domains of proteins. While some of these lipophilic dyes have been used as stains for lipid membranes, they have not previously been disclosed for the use of a selective myelin stain. The present staining method distinguishes between lipid membranes and myelin, wherein lipid membranes may be faintly visible but are clearly distinguished from myelin after labeling.

Thus, the present invention, includes with out limit lipophilic dyes such as cyanine dyes, merocyanine dyes, styryl dyes and carbazolylvinyl dyes and any dye disclosed in U.S. Pat. Nos. 6,579,718; 5,616,502; 5,436,134; 5,656,449; 5,658,751; 6,004,536; 4,883,867 and 4,957,870. It is understood that a larger number of lipophilic dyes have been previously disclosed and that the invention is not limited to those dyes disclosed in the above patent references but includes all known lipophilic cyanine dyes, merocyanine dyes, styryl dyes and carbazolylvinyl dyes and those invented in the future.

Cyanine, styryl, carbazolylvinyl, and merocyanine dyes are a diverse group of dyes that comprise a quaternary nitrogen heterocycle linked to an electron pair-donating moiety by an alkylene or polyalkylene bridge. Thus, in an exemplary embodiment, the present lipophilic dyes are represented by the general formula A-B-E wherein A is a nitrogen heterocycle, B is a bridge moiety; and E is an electron pair accepting moiety that comprises either a carbonyl or nitrogen atom.

In an exemplary embodiment A is a quaternized nitrogen heterocycle where the quaternizing group R² that is represented by the formulas:

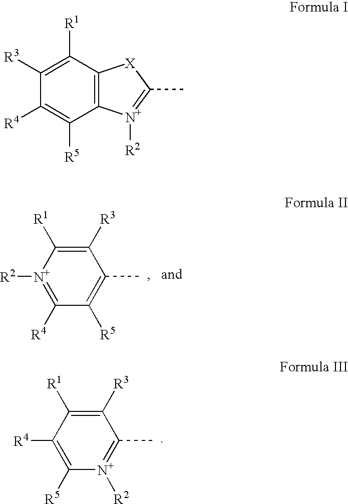

The quarternizing nitrogen substituent R² is alkyl, substituted alkyl, sulfoalkyl, substituted sulfoalkyl, aminoalkyl or substituted aminoalkyl. R² is typically a sulfoalkyl, aminoalkyl or a substituted aminoalkyl wherein the amino group is substituted with an alkyl, aminoalkyl or sulfoalkyl.

In an exemplary embodiment R² includes at least one nitrogen heteroatom, preferably wherein the nitrogen atom is a dialkylamino or a trialkylammonium substituent, and where the alkyl substituents are methyl or ethyl. In another embodiment, R² is —CH₃, or CH₂CH₃, or R² is a C₃-C₂₂ alkyl chain that is linear or branched, saturated or unsaturated, and that is optionally substituted one or more times by hydroxy, carboxy, sulfo, amino, amino substituted by 1-2 C₁-C₆ alkyls, or ammonium substituted by 1-3 C₁-C₆ alkyls. In one aspect R² is a C₃-C₁₂ alkyl chain that is linear and saturated, and substituted at its free terminus by hydroxy, carboxy, sulfo, amino, amino substituted by 1-2 C₁-C₆ alkyls, or ammonium substituted by 1-3 C₁-C₆ alkyls. In another aspect R² is a C₃-C₄ alkyl that is substituted once by sulfo or carboxy.

Alternatively, the nitrogen atoms of R² form either one or two saturated 5- or 6-membered rings in combination with other C or N atoms in R², such that the resulting rings are pyrrolidines, piperidines, piperazines or morpholines.

The aromatic substitutents R¹, R³, R⁴, R⁵ are independently hydrogen, halogen, substituted halogen, alkyl, substituted alkyl, sulfoalkyl, alkoxy, substituted alkoxy, amino, substituted amino, aminoalkyl or substituted aminoalkyl. Alternatively, the aromatic ring can be fused to additional rings wherein a member independently selected from; R¹ in combination with R³; and R⁴ in combination with R⁵; together with the atoms to which they are joined, form a ring which is a 5-, 6- or 7-membered cycloalkyl, a 5-, 6- or 7-membered heterocycloalkyl, a 5-, 6- or 7-membered aryl or a 5-, 6- or 7-membered heteroaryl (yielding a benzo-substituted pyridinium, or quinolinium moiety).

The additional ring on the quinolinium that is thereby formed is optionally and independently substituted one or more times by halogen, alkyl, perfluoroalkyl, alkoxy, amino, or amino substituted by alkyls. Additionally, the quinolinium ring is optionally substituted by an additional fused 6-membered aromatic ring (yielding a naphtho-substituted pyridinium, or a benzoquinoline), that is also optionally and independently substituted one or more times by halogen, alkyl, perfluoroalkyl, alkoxy, amino, or amino substituted by alkyls. Typically, R¹ and R³ are hydrogen, or form a substituted or unsubstituted benzo moiety.

In the benzazole ring (Formula I), the ring fragment X is O, S, NR⁵, or CR¹¹R¹² wherein R⁵ is disclosed above and R¹¹ and R¹² are independently hydrogen, halogen, phenyl, substituted phenyl, substituted halogen, alkyl, or substituted alkyl or R¹¹ and R¹² in combination form a 5- or 6-membered ring. When X is CR¹¹R¹², R¹¹ and R¹² are typically hydrogen. Typically X is O or S, more typically X is O.

B is a covalent bridge that is an alkylene or polyalkylene covalent linkage that is generally referred to as a methine bridge. B has the formula —(CR¹═CR ¹²)ₙ— wherein R¹¹ and R¹² are independently hydrogen, halogen, phenyl, substituted phenyl, substituted halogen, alkyl, or substituted alkyl. In one aspect, R¹¹ and R¹² are hydrogen.

n is 1, 2, or 3 and determines how many conjugated alkenyl moieties are joined to form the bridge. The spectral properties of the resulting dye are highly dependent upon the length of the bridge moiety, with the excitation and emission wavelengths shifting to longer wavelengths with the addition of each alkenyl moiety. Thus, when selecting dyes, compounds with longer methine bridges, wherein n is 2 or 3 will typically have a longer emission wavelength than those compounds wherein n is 1.

A wide variety of electron pair-donating groups are known that stabilize the formally positive charge of the quaternary nitrogen heterocycle by resonance. Suitable electron pair-donating groups include dialkylaminophenyl, dialkylaminonaphthyl, electron-rich heterocycles and acyclic moieties containing electron pair-donating groups.

In an exemplary embodiment E is an aromatic heterocyclic substituent or activated methylene substituent. In a further embodiment E is represented by the formula:

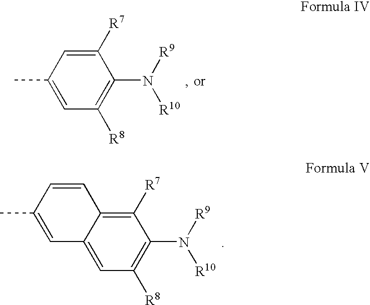

The aromatic substituents R⁷ and R⁸ of Formula IV and V are independently hydrogen, halogen, substituted halogen, alkyl, substituted alkyl, sulfoalkyl, amino, substituted amino, aminoalkyl or substituted aminoalkyl. In an exemplary embodiment R⁷ and R⁸ are hydrogen.

The amino substituents R⁹ and R¹⁰ are independently alkyl, substituted alkyl, sulfoalkyl, aminoalkyl or substituted aminoalkyl. In one embodiment R⁹ and R¹⁰ are C₁-C₁₈ alkyls that are linear, branched, saturated or unsaturated, and are optionally substituted one or more times by halogen, hydroxy or alkoxy. In a further aspect, R⁹ and R¹⁰ are each linear C₄-C₈ alkyls, preferably R⁹ and R¹⁰ are C₅-C₇ alkyls. Alternatively, R⁹ and R¹⁰ in combination form a 5- or 6-membered ring; R⁹ and R⁷ in combination for a 5- or 6-membered ring or R¹⁰ and R⁸ in combination form a 5- or 6-membered ring. In one embodiment the formed ring contains an oxygen heteroatom.

In a particularly preferred embodiment at least one of R⁹ and R¹⁰ or both contain a lipophilic alkyl moiety wherein the alkyl portion contains at least four carbons.

In an exemplary embodiment the lipophilic dyes are represented by the general formula

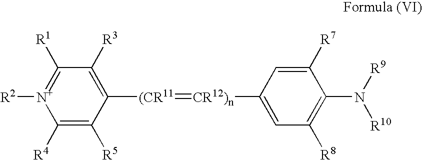

Wherein R¹, R², R³, R⁴, R⁵, R⁷, R⁸, R⁹, R¹⁰, R¹¹ and R¹² are defined above.

A particularly preferred dye of the present invention is Compound 1 and 2 that is represented by the structure: 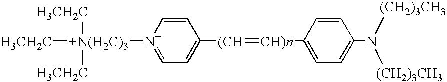
wherein n is 1 (Compound 1) or 3 (Compound 2).

In another aspect, a preferred dye of the present invention is Compound 4 that is represented by the formula:

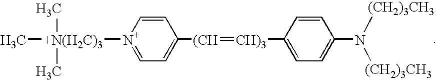

Unexpectedly Compound 3, was not selective for myelin (See, Example 4)

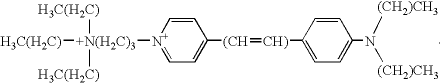

In a further embodiment, wherein R⁴ and R⁵ form a 6-membered fused ring, the lipophilic dyes are represented by the formula:

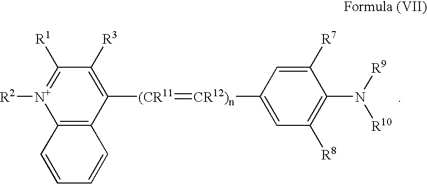

Wherein R¹, R², R³, R⁷, R⁸, R⁹, R¹⁰, R¹¹ and R² are defined above.

In another exemplary embodiment E is a substituted or unsubstituted carbazolyl moiety attached at its 3-position to B that is an ethenyl (vinyl) or polyethenyl/alkylene or polyalkylene bridging moiety. In one aspect E is represented by the formula:

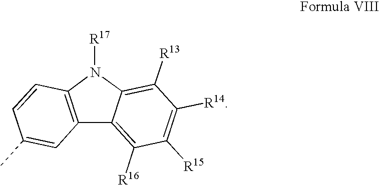

The aromatic substituents R¹³, R¹⁴, R¹⁵, and R¹⁶ are independently hydrogen, halogen, substituted halogen, alkyl, substituted alkyl, sulfoalkyl, alkoxy, substituted alkoxy, amino, substituted amino, aminoalkyl or substituted aminoalkyl.

The nitrogen substituent R¹⁷ of Formula VI is alkyl, substituted alkyl, phenyl, substituted phenyl, amino alkyl, or substituted aminoalkyl. In one embodiment R¹⁷ is methyl, ethyl or phenyl. In another embodiment R¹⁷ is a sulfoalkyl or an aminoalkyl wherein the amino group is optionally substituted by an alkyl group or an aminoalkyl group.

In an exemplary embodiment the lipophilic dyes are represented by the general formula:

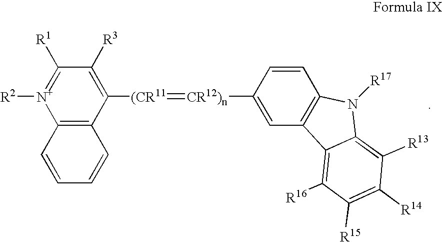

Wherein R¹, R², R³, R¹¹, R¹², R¹³, R¹⁴, R¹⁵, R¹⁶ and R¹⁷ are defined above.

Syntheses of many of the preferred embodiments of the dyes are well documented including in the following references (U.S. Pat. Nos. 5,616,502; 6,579,718; Grinvald et al., BIOPHYS. J. 39, 301 (1982); Leslie Loew (Loew et al., J. ORG. CHEM. 49, 2546 (1984); Brooker et al. (J. AM. CHEM. SOC. 73, 5326 (1951)).

Methods of Use

The present invention utilizes the lipophilic dyes described above to stain myelin in samples.

In an exemplary embodiment, the present method comprises:
a) contacting the sample with a lipophilic fluorescent dye that selectively associates with myelin to prepare a labeling mixture;
b) incubating the labeling mixture for a sufficient amount of time for the dye to associate with the myelin to form an incubated sample;
c) illuminating the incubated sample with an appropriate wavelength to form an illuminated sample; and
d) observing the illuminated sample whereby the myelin is detected.

A particular dye of this invention is generally selected for a particular assay using one or more of the following criteria: sensitivity to myelin, insensitivity to the presence of nucleic acids, insensitivity to lipid membranes, dynamic range, photostability, staining time, and spectral properties. The sensitivity and dynamic range of the dyes is determined using the procedures of Example 1.

The present lipophilic dyes are prepared according to methods generally known in the art. The dyes are generally soluble in water and aqueous solutions having a pH greater than or equal to about 6. Stock solutions of pure dyes, however, are typically dissolved in organic solvent before diluting into aqueous solution or buffer. Alternatively the dyes are stored in lyophilized form and then diluted in an organic solvent. Preferred organic solvents are aprotic polar solvents such as DMSO, DMF, N-methylpyrrolidone, acetone, acetonitrile, dioxane, tetrahydrofuran and other nonhydroxylic, completely water-miscible solvents.

In general, the amount of dye in the dye staining solution that is added to a sample is the minimum amount required to yield detectable staining in the sample within a reasonable time, with minimal background fluorescence or undesireable staining.

The exact concentration of dye to be used is dependent upon the experimental conditions and the desired results, and optimization of experimental conditions is typically required to determine the best concentration of dye to be used in a given application. The concentration of dye present in the dye solution typically ranges from nanomolar to micromolar. The required concentration for the dye solution is determined by systematic variation in dye concentration until satisfactory dye staining is accomplished.

The lipophilic dyes described above, specifically or generically, are considered part of the invention to be used in the present staining method. For fluorescence detection, dye concentrations are typically greater than 50 nM and less than 5 μM; preferably greater than about 250 nM and less than or equal to about 2.5 μM; more preferably about 400-600 nM. In one aspect the concentration of the dye in the staining solution is 500 nM. Although concentrations below and above these values likewise result in detectable staining for myelin, depending on the sensitivity of the detection method, dye concentrations greater than about 10 μM generally lead to some quenching of the fluorescence signal.

To make a staining solution to combine with the sample, the selected dye is typically first dissolved in an organic solvent, such as water, DMSO, DMF or methanol, usually to a dye concentration of 100-500 μM. In an exemplary embodiment, the dye is stored as a concentrate of about 150 μM in water.

This concentrated stock solution is then generally diluted in an aqueous buffer. Buffering components include but are not limited to, 50-100 mM formate buffer, pH 4.0, sodium citrate, pH 4.5, sodium acetate, pH 5.0, MES, pH 6.0, imidazole, pH 7.0, HEPES, pH 6.8, Tris acetate, pH 8.0, Tris-HCl, pH 8.5, Tris borate, pH 9.0 and sodium bicarbonate, pH 10, phosphate buffered saline (PBS), pH 7.0. In an exemplary embodiment, the stock dye solution is diluted in PBS at a working concentration of about 500 nM.

The present lipophilic dyes, in the form of a staining solution, are contacted with the sample to form a labeling mixture. The sample is typically a tissue section that is believed to comprise myelin. In one embodiment, this tissue section is a brain tissue section, however any tissue section that is thought to contain myelin can be used.

The sample is prepared in such a way as to facilitate contact between the myelin and the staining solution. In this instance, the tissue sections are typically immobilized on a solid or semisolid support, such as but not limited to, a polymeric membrane, within a polyacrylamide gel, within an agarose gel, on a polymeric membrane, on a glass slide or on a microarray. In one embodiment, the sample is immobilized on a glass slide using standard techniques of the art. The sample includes any tissues or cultured axons that contain myelin including brain tissue and peripheral axons.

The labeling mixture is incubated for a sufficient amount of time to allow the present dye to associate with the present myelin. The labeled sample mixture is typically incubated for less than about 12 hours, typically less than about 8 hours, more typically less than about 4 hours. In one aspect the sample and staining solution are incubated less than about 1 hour and in a further aspect the sample and staining solution are incubated about 20 minutes.

In an exemplary embodiment, the present method stains and detects myelin in brain tissue sections that have been immobilized on a glass slide. In this instance a protocol for labeling brain cryosections comprises:
1. Rehydrate and/or Permeabilize. Bring tissue sections on slides to room temperature, then rehydrate in either PBS (phosphate-buffered saline, 0.05M, pH 7.4) or PBT (PBS+0.2% Triton X-100) for at least 20 minutes. Permeabilization with Triton X-100 is not necessary for labeling using the present lipophilic dyes, but is likely necessary for any other counterstains or antibodies to be used.
2. Prepare Label Solution. Prepare the labeling solution by diluting the stock solution 300-fold with PBS to make a final recommended concentration of 500 nM dye.
3. Label Step. Flood the section with labeling solution and stain for 20 minutes at room temperature.
4. Wash. When labeling is complete, remove solution, rinse in PBS, and wash 3×10 minutes with PBS.
5. Counterstaining and Mounting. At this point tissues can be counterstained as necessary, or mounted with an aqueous antifade mounting medium such as ProLong or ProLong Gold reagent.

Counterstains, or additional detection reagents include, but are not limited to, an antibody, a nucleic acid stain, an ion indicator, a cytoskeleton stain, an extracellular matrix stain or an organelle stain. Organelle stains, include but are not limited to, stains that are selective for mitochondria, lysozymes, nucleus, golgi, or endoplastic reticulum (ER). In one aspect the additional detection reagent is DAPI, which binds specifically to nucleic acid. In another aspect, the additional detection reagent is NeuroTrace Red Nissl Stain, which selectively labels neuron cell bodies by binding to the rough endoplasmic reticulum of neuronal perikarya and dendrites (the “Nissl substance”), and is red-fluorescent. Alternatively, NeuroTrace Green Nissl Stain or Neutral Red is used. Additional detection reagents that can be used instead of NeuroTrace Red or Green Nissl Stain are cresyl violet, methylene blue, safranin-O, and toluidine blue-O. Alternatively, a sample may be stained with more than one additional detection reagent, in addition to the present myelin stain.

The additional detection reagent can be added before, during or after the sample has been incubated with the present dyes. In an exemplary embodiment the present dye is combined with an additional detection reagent to form a combined staining solution. The combined staining solution is added to the sample wherein myelin and a different discrete molecule and/or locations are stained within the same sample, See Example 2 and 3.

Illumination

At any time after or during staining, the sample is illuminated with a wavelength of light selected to give a detectable optical response, and observed with a means for detecting the optical response. Equipment that is useful for illuminating the dye compounds of the invention includes, but is not limited to, hand-held ultraviolet lamps, mercury arc lamps, xenon lamps, lasers and laser diodes. These illumination sources are optionally integrated into laser scanners, fluorescence microplate readers, standard or minifluorometers, or chromatographic detectors.

The optical response is optionally detected by visual inspection, or by use of any of the following devices: CCD cameras, video cameras, photographic film, laser-scanning devices, fluorometers, photodiodes, quantum counters, epifluorescence microscopes, scanning microscopes, fluorescence microplate readers, or by means for amplifying the signal such as photomultiplier tubes. A detectable optical response means a change in, or occurrence of, an optical signal that is detectable either by observation or instrumentally. Typically the detectable response is a change in fluorescence, such as a change in the intensity, excitation or emission wavelength distribution of fluorescence, fluorescence lifetime, fluorescence polarization, or a combination thereof.

In particular, present lipophilic dyes can be selected that possess excellent correspondence of their excitation band with the 488 nm band of the commonly used argon laser or emission bands which are coincident with preexisting filters

Kits

Suitable kits for selectively detecting myelin in a sample also form part of the invention. Such kits can be prepared from readily available materials and reagents and can come in a variety of embodiments. The contents of the kit will depend on the design of the assay protocol or reagent for detection or measurement. All kits will contain instructions, appropriate reagents, and staining solution. Typically, instructions include a tangible expression describing the reagent concentration or at least one assay method parameter such as the relative amounts of reagent and sample to be added together, maintenance time periods for reagent/sample admixtures, temperature, buffer conditions and the like to allow the user to carry out any one of the methods or preparations described above.

Thus, in an exemplary embodiment, a kit comprises a lipophilic dye either in a stock concentrate or a ready to use present staining solution. In a further aspect the kits contain additional detection reagents. In this instance additional detection reagents include, but are not limited to, an antibody, a nucleic acid stain, an ion indicator, a cytoskeleton stain, an extracellular matrix stain or an organelle stain. Organelle stains, include but are not limited to, stains that are selective for mitochondria, lysozymes, nucleus, golgi, or endoplastic reticulum (ER). In one aspect a kit of the present invention contains a nuclear stain and an organelle stain.

A detailed description of the invention having been provided above, the following examples are given for the purpose of illustrating the invention and shall not be construed as being a limitation on the scope of the invention or claims.

### EXAMPLES

### Example 1

### Fluoromyelin Label Pattern Comparisons

A. Anti-Myelin Basic Protein (MBP).

Use of this antibody allows for specific labeling with which to compare the present lipophilic dyes and chromogenic method label patterns. Comparisons were run on 16 μm mouse brain cryosections, using mid-sagittal sections through whole brain as well as various cross-sections through the diencephalon. Tissue was sectioned on a cryostat and mounted on Plus slides and stored at −80° C. Upon use, tissue sections were brought to room temperature and rehydrated in PBT (0.5 M phosphate-buffered saline containing 0.2% Triton X-100 and 0.2% BSA) for 30 minutes. Sections were blocked for 1 hour in 5% normal goat serum/3% BSA/PBT, then labeled overnight at 4° using 5 μg/ml rat anti-MBP primary antibody from Chemicon (MAB386). After washing in PBT, tissues were washed in Image-iT FX signal enhancer (136933, Molecular Probes, Inc., Eugene, Oreg.) for 30 minutes, then washed again in PBT. Sections were incubated in Alexa Fluor 488 goat anti-rat secondary antibody (A11006, Molecular Probes, Inc.) for 2 hours, washed in PBT, and mounted in ProLong antifade solution (P7481, Molecular Probes, Inc.). Images were collected on a Nikon E800 epifluorescence upright microscope using a Princeton MicroMax cooled CCD digital camera, MetaMorph acquisition software, and Adobe Photoshop image analysis software (Ex 495/Em 519, FITC Filter Set). Results were specific to expected label pattern, including individualized axons and axon bundles, with very little nonspecific background.

Sagittal sections showed excellent labeling of general axon tracts, the corpus collosum in the cerebrum, and arbor vitae in the cerebellum, for example. The diencephalic cross section showed, as example, labeling of tracts in the fornix, fornix superior, capsula interna, and striatum.

B. Chromogenic Labeling Techniques.

Other than antibody labeling techniques, myelin labeling is traditionally performed using long and complicated chromogenic (non-fluorescent) dye protocols. One of the most common is the Loyez Technique. A more recent, and more specific method is Schmued's Gold Chloride Technique. These two methods were used for comparison to the present dyes.
I. Schmued's Gold Chloride Technique. Based on a method by Laurence C. Schmued [1990. A rapid, sensitive histochemical stain for myelin in frozen brain sections. J. Histochem Cytochem. 38(5): 717-720]. Tissues were rehydrated in PBT for 30 minutes, then labeled for 3.5 hours in 0.2% gold chloride in 0.02 M neutral phosphate buffer with 0.9% sodium chloride. After a water rinse, tissues were post-fixed for 5 minutes in 2.5% sodium thiosulfate, rinsed in running tap water for 30 minutes, and mounted in mowiol. Images were collected using transmitted light on the system outlined in part A of this example. Myelin was labeled a brown-red color. Results showed comparable staining to anti-MBP, as outlined in part A, for both sagittal and cross sections. The only significant difference was the lack of single axon labeling and a slight increase in non-specific background.
II. Loyez Technique. Adapted from H. C. Cook. 1974. Manual of Histological Demonstration Techniques. London: Butterworths. pp. 161-162. Tissues were rehydrated and permeabilized 30 minutes in PBT, then treated with 4% iron alum overnight at room temperature. After a water wash, tissues were stained overnight in a 10% alcoholic haematoxylin solution with lithium carbonate at room temperature. After two 10-minute water washes, tissues were destained for approximately 20 seconds in 4% iron alum, and washed in water. After a final wash in borax-ferricyanide to colorize the myelin blue-black, tissues were washed in water and mounted in mowiol. Images were scanned as in section B. I. Of this example. Results showed myelin colorized blue-black, with a label pattern comparable to Schmued's Gold Chloride Technique. Like the Schmued's technique, there was loss of single-axon labeling seen with anti-MBP, and a higher non-specific background label. Dim nuclear labeling was also apparent in areas. Results were the same for diencephalic sections as well as sagittal sections.
   C. Myelin Staining with Compound 1:

Mouse brain cryosections were rehydrated for 30 minutes in PBT, then labeled for 20 minutes with 500 nM of Compound 1 in PBS. After labeling, sections were washed 3 times with PBT, then mounted in ProLong antifade mounting medium (Molecular Probes, Inc.). Imaging was performed as in section A. The dye fluoresced at expected wavelengths, similar to FITC (Ex 479/Em 594). The label pattern showed myelin staining in cross sections and sagittal sections that was most comparable to Schmued's Gold Chloride Technique, with specific myelin labeling in expected axon bundles and tracts. Like the two chromogenic methods, single axon labeling was not seen, as it had been with anti-MBP, and some non-specific background labeling was visualized.

D. Myelin Staining with Compound 2:

Mouse brain cryosections were rehydrated for 30 minutes in PBT, then labeled for 20 minutes with 500 nM of Compound 2 in PBS. After labeling, sections were washed 3 times with PBT, then mounted in ProLong antifade mounting medium (Molecular Probes, Inc.). Imaging was performed as in section A of this example. The dye was visible using a standard TRITC filter set (Ex 558/Em 734). The label pattern showed myelin staining in cross sections and sagittal sections that was most comparable to Schmued's Gold Chloride Technique, with specific myelin labeling in expected axon bundles and tracts. Like the two chromogenic methods, single axon labeling was not seen, as it had been with anti-MBP, and some non-specific background labeling was visualized.

### Example 2

### Detection of Myelin in Combination with Antibodies

To test whether use of Compound 1 interrupts antibody binding, Compound 1 was used in conjunction with anti-GFAP on mouse brain sections. A 16 μm mouse brain cryosection, cross-section through the rostral mesencephalon, was rehydrated and permeabilized in PBT (0.05 M phosphate buffered saline with 0.2% Triton X-100 and 0.2% BSA) for 20 minutes, the washed for 30 minutes with Image-iT FX Signal Enhancer (Molecular Probes, Inc.) to block non-specific Alexa Fluor dye binding to myelin. After washing in PBT, the section was blocked for 1 hour in 5% normal goat serum/0.2% Triton X-100/3% BSA, then incubated overnight at 4° C. in 5 μg/ml mouse anti-GFAP (glial fibrillary acidic protein) from Molecular Probes (A21282) in blocking buffer. Sections were washed well in PBT, then incubated in Alexa Fluor 680 goat anti-mouse secondary antibody (A21057, Molecular Probes, Inc.) for 2 hours. After washing in PBT, sections were labeled in 500 nM of Compound 1 for 20 minutes with 0.1 μg/ml DAPI. After a final wash in PBT, sections were mounted in Prolong antifade mountant (Molecular Probes, inc.). Images were collected on a Nikon E800 epifluorescence upright microscope using a Princeton MicroMax cooled CCD digital camera, MetaMorph acquisition software, and Adobe Photoshop image analysis software. Results showed that all dyes fluoresced at expected wavelengths (Ex 479/Em 594), with a label pattern specific to expected structures: anti-GFAP was specific to astrocytes, and Compound 1 labeled axon tracts and bundles in, for instance, the commissural fornicis dorsalis, alveus hippocampi, fasciculus mammillotegmentalis. Thus, in this system, there was no inhibition of staining pattern by either label.

### Example 3

### Compound 1 and 2 Use in Combination with NeuroTrace Nissl Dyes and DAPI

Compound 1 and 2 were tested with NeuroTrace Nissl stains and DAPI in the same label solution in order to determine if a kit could be made for this purpose, with multiple components.

A. Compound 1, NeuroTrace Red Nissl Stain, and DAPI

A 16 μm mouse brain cryosection, cross section through diencephalon, was rehydrated for 20 minutes in PBT (0.5 M phosphate-buffered saline containing 0.2% Triton X-100 and 0.2% BSA). The three dyes were mixed into the final stain solution (in PBS), such that there was a concentration of 500 nM of Compound 1, 1:300 dilution of NeuroTrace Red Nissl Stain, and 0.1 μg/ml DAPI. Sections were labeled in this combination for 20 minutes, then washed well in PBT prior to mounting in Prolong antifade mounting medium (Molecular Probes, Inc.). Images were collected on a Nikon E800 epifluorescence upright microscope using a Princeton MicroMax cooled CCD digital camera, MetaMorph acquisition software, and Adobe Photoshop image analysis software. Results showed each dye to fluoresce in expected wavelengths (Compound 1: Ex 479/Em 594; NeuroTrace Red Nissl: Ex 530/Em 615; DAPI: Ex 358/Em 461), with label patterns that corresponded to expected results: Compound 1 labeled, for instance, tracts in the fornix, fornix superior, capsula interna, and striatum; NeuroTrace Nissl stain labeled neuron cell bodies, and DAPI labeled nuclei. There was no evidence of disruption of any of the three labels.

B. Compound 2, NeuroTrace Green Nissl Stain, and DAPI

A 16 μm mouse brain cryosection, cross section through diencephalon, was rehydrated for 20 minutes in PBT (0.5M phosphate-buffered saline containing 0.2% Triton X-100 and 0.2% BSA). The three dyes were mixed into the final stain solution (in PBS), such that there was a concentration of 500 nM Compound 2, 1:300 dilution of NeuroTrace Red Nissl Stain, and 0.1 μg/ml DAPI. Sections were labeled in this combination for 20 minutes, then washed well in PBT prior to mounting in Prolong antifade mounting medium (Molecular Probes, Inc.). Images were collected on a Nikon E800 epifluorescence upright microscope using a Princeton MicroMax cooled CCD digital camera, MetaMorph acquisition software, and Adobe Photoshop image analysis software. Results showed each dye to fluoresce in expected wavelengths (Compound 2: Ex 558/Em 734; NeuroTrace Green Nissl: 500/525; DAPI 358/461), with label patterns that corresponded to expected results: Compound 2 labeled, for instance, tracts in the fornix, fornix superior, capsula interna, and striatum; NeuroTrace Nissl stain labeled neuron cell bodies, and DAPI labeled nuclei. There was no evidence of disruption of any of the three labels.

### Example 4

### Brain Tissue Staining with Compound 3

12 μm Thick mouse brain cryosections were rehydrated in phosphate-buffered saline (PBS, 0.05 M, pH 7.4) for 20 minutes, then labeled with 1 μM or 500 nM of Compound 3 in PBS. Sections were washed 3×10 minutes in PBS, then mounted in Prolong Gold antifade mounting medium. Samples were compared to sections labeled with Compound 1 and Compound 2 as controls. While the control dyes worked as expected, with selective myelin labeling, Compound 3 showed no apparent labeling of myelin at either concentration.

### Example 5

### Compound 4 Basic Label Pattern and Concentration

12 μm thick mouse brain cryosections were rehydrated in phosphate-buffered saline (PBS, 0.05M, pH 7.4) for 20 minutes, then labeled with 1 μM or 500 nM Compound 4 in PBS. Sections were washed 3×10 minutes in PBS, then mounted in Prolong Gold antifade mounting medium. Samples were compared to sections labeled with Compound 1 and Compound 2 as controls. Images were collected on a Nikon E800 epifluorescence upright microscope using a Princeton MicroMax cooled CCD digital camera, MetaMorph acquisition software, and Adobe Photoshop image analysis software. Compound 4 showed highly selective labeling of myelin, comparable in label pattern to both Compound 1 and Compound 2, with spectral qualities comparable to Compound 2. 500 nM concentration was sufficient for labeling with minimal background, as seen with the control dyes.

### Example 6

### Compound 4 on Cross- and Sagittal-Sectioned Brain with NeuroTrace Green Nissl Stain

12 μm thick mouse brain cryosections, either cross-sectioned through the mesencephalon or sagittal sections, were rehydrated and permeabilized for 20 minutes in PBT (PBS with 0.2% Triton X-100 and 0.2% BSA) then labeled 30 minutes with 500 nM Compound 4 in PBS. Sections were washed 3×10 minutes with PBT then labeled 20 minutes with NeuroTrace Green Nissl Stain (diluted 1:300 from stock). Sections were washed 3×5 minutes in PBS, labeled 1.5 minutes with 0.2 μg/mL DAPI, washed 3×5 minutes in PBS again, and mounted in Prolong Gold antifade mounting medium. Samples were compared to sections labeled with Compound 1 and Compound 2 as controls. Images were collected on a Nikon E800 epifluorescence upright microscope using a Princeton MicroMax cooled CCD digital camera, MetaMorph acquisition software, and Adobe Photoshop image analysis software. Results showed label pattern comparable to the control dyes. Compound 4 was shown to be compatible with NeuroTrace Green Nissl Stain labeling and DAPI labeling, providing three-color labeling.

### Example 7

### Demyelinated Mouse Model Test

To illustrate the use of the present dyes for studying myelin diseases such as multiple sclerosis, adrenoleukodystrophy, Guillain-Barre syndrome, and others, Compound 1 was compared against rat anti-myelin basic protein (anti-MBP) primary antibody on control mouse brains versus shiverer mouse brain. Shiverer mice (JAX GEMM Strain C3Fe.SWV-Mbp^{shi}/J), a homozygous spontaneous mutation mouse line which exhibits demyelinated brain tissue, were perfuse-fixed and dissected along with control mice (JAX C57BU6J). Brain tissues were cryosectioned at 16 μm and mounted on PLUS slides. Control and shiverer tissues were treated either with anti-MBP primary and Alexa Fluor 488 goat anti-rat secondary or Compound 1. Tissues treated with the antibody were rehydrated and permeabilized in PBT (0.05 M phosphate buffered saline with 0.2% Triton X-100 and 0.2% bovine serum albumin (BSA)) for 20 minutes, then blocked in 5% normal goat serum/3% BSA/PBT for 1 hour. Tissues were then incubated overnight at 4° C. with 5 μg/mL rat anti-MBP (Chemicon, MAB386) in blocking solution. After washing 3×10 minutes in PBT, they were then incubated for 2 hours with 5 μg/mL Alexa Fluor 488 goat anti-rat secondary at room temperature. After further washing 3×10 minutes in PBT, tissues were counterstained with 0.2 μg/ml DAPI, washed 3×5 minutes in PBT, and mounted with ProLong antifade mounting medium (Invitrogen P36930). Tissues that were labeled with Compound 1 were rehydrated and permeabilized as above, then labeled 20 minutes with 500 nM Compound 1 and 0.2 μg/mL DAPI (together) in PBT. Tissues were washed 3×10 minutes PBT, then mounted as above. Images were collected on a Nikon E800 epifluorescence upright microscope using a Princeton MicroMax cooled CCD digital camera, an Omega XF100-2 FITC filter set, MetaMorph acquisition software, and Adobe Photoshop image analysis software. Results showed that both the anti-MBP and Compound 1 labeled myelin in control mice, with anti-MBP giving the expected greater resolution of individual axons, though Compound 1 staining was twice as bright. Myelin labeling was negative, as expected, for shiverer mice for both anti-MBP and Compound 1, illustrating that the present dyes work as well as anti-MBP for demyelination determination. Compound 1, however, exhibited slight background labeling not seen with anti-MBP due to general membrane labeling.

What is claimed is:

1. A method for detecting myelin in a sample, wherein the method comprises:
a. contacting the sample with a lipophilic fluorescent dye that selectively associates with myelin to form a labeling mixture;
b. incubating the labeling mixture for a sufficient amount of time for the dye to associate with the myelin to form an incubated sample;
c. illuminating the incubated sample with an appropriate wavelength to form an illuminated sample; and
d. observing the illuminated sample whereby the myelin is detected.

2. The method according to claim 1, wherein the sample is a tissue section.

3. The method according to claim 2, wherein the tissues section is a brain tissue section.

4. The method according to claim 1, further comprising washing the incubated sample to remove unbound dye.

5. The method according to claim 1, further comprising contacting the sample with an additional detection reagent.

6. The method according to claim 5, wherein the additional detection reagent is an antibody, a nucleic acid stain, an ion indicator, a cytoskeleton stain, an extracellular matrix stain or an organelle stain.

7. The method according to claim 6, wherein the organelle stain is selective for mitochondria, lysozymes, nucleus, golgi, or endoplastic reticulum (ER).

8. The method according to claim 1, wherein the lipophilic dye is a merocyanine dye, a cyanine dye, a styryl dye or a carbazolylvinyl dye

9. The method according to claim 1, wherein the lipophilic dye has the general formula:
A-B-E
wherein A is a nitrogen heterocycle;
B is a bridge moiety; and
E is an electron pair accepting moiety that comprises either a carbonyl or nitrogen atom.

10. The method according to claim 9, wherein the lipophilic dye is a cyanine dye.

11. The method according to claim 9, wherein the lipophilic dye is a merocyanine dye

12. The method according to claim 9, wherein A is 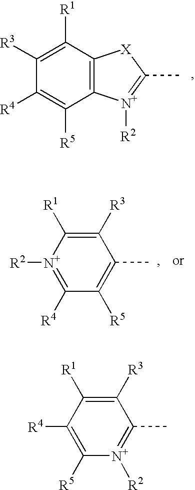
wherein,
R¹ is hydrogen, halogen, substituted halogen, alkyl, substituted alkyl, sulfoalkyl, alkoxy, substituted alkoxy, amino, substituted amino, aminoalkyl or substituted aminoalkyl;
R² is alkyl, substituted alkyl, sulfoalkyl, aminoalkyl, substituted aminoalkyl, sulfoalkyl or substituted sulfoalkyl;
R³ is hydrogen, halogen, substituted halogen, alkyl, substituted alkyl, sulfoalkyl, alkoxy, substituted alkoxy, amino, substituted amino, aminoalkyl or substituted aminoalkyl;
R⁴ is hydrogen hydrogen, halogen, substituted halogen, alkyl, substituted alkyl, sulfoalkyl, alkoxy, substituted alkoxy, amino, substituted amino, aminoalkyl or substituted aminoalkyl;
R⁵ is hydrogen hydrogen, halogen, substituted halogen, alkyl, substituted alkyl, sulfoalkyl, alkoxy, substituted alkoxy, amino, substituted amino, aminoalkyl or substituted aminoalkyl;
or a member independently selected from;
R¹ in combination with R³;
R³ in combination with R⁴; and
R⁴ in combination with R⁵;
together with the atoms to which they are joined, form a ring which is a 5-, 6- or 7-membered cycloalkyl, a 5-, 6- or 7-membered heterocycloalkyl, a 5-, 6- or 7-membered aryl or a 5-, 6- or 7-membered heteroaryl; and
X is O, S, NR⁵, or CR¹¹R¹² wherein R¹¹ is hydrogen, halogen, phenyl, substituted phenyl, substituted halogen, alkyl, or substituted alkyl; R¹² is hydrogen, halogen, phenyl, substituted phenyl, substituted halogen, alkyl, substituted alkyl; or R¹¹ and R¹² in combination form a 5- or 6-membered ring;
B is a covalent bridge having the formula —(CR¹¹═CR¹²)ₙ—;
wherein R¹¹ is hydrogen, halogen, phenyl, substituted phenyl, substituted halogen, alkyl, or substituted alkyl;
R¹² is hydrogen, halogen, phenyl, substituted phenyl, substituted halogen, alkyl, or substituted alkyl;
or R¹¹ and R¹² in combination form a 5- or 6-membered ring;
n is 1, 2or 3;
E is 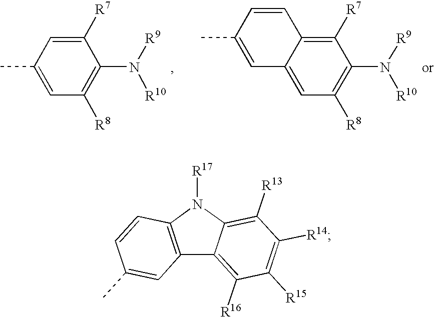
wherein R⁷ is hydrogen, halogen, substituted halogen, alkyl, substituted alkyl, sulfoalkyl, amino, substituted amino, aminoalkyl or substituted aminoalkyl;
R⁸ is hydrogen, halogen, substituted halogen, alkyl, substituted alkyl, sulfoalkyl, amino, substituted amino, aminoalkyl or substituted aminoalkyl;
R⁹ is alkyl, substituted alkyl, sulfoalkyl, aminoalkyl or substituted aminoalkyl;
R¹⁰ is alkyl, substituted alkyl, sulfoalkyl, aminoalkyl or substituted aminoalkyl; or
R⁹ and R¹⁰ in combination form a 5- or 6-membered ring, R⁹ and R⁷ in combination for a 5- or 6-membered ring or R¹⁰ and R⁸ in combination form a 5- or 6-membered ring;
R¹³ is hydrogen, halogen, substituted halogen, alkyl, substituted alkyl, sulfoalkyl, alkoxy, substituted alkoxy, amino, substituted amino, aminoalkyl or substituted aminoalkyl;
R¹⁴ is hydrogen, halogen, substituted halogen, alkyl, substituted alkyl, sulfoalkyl, alkoxy, substituted alkoxy, amino, substituted amino, aminoalkyl or substituted aminoalkyl;
R¹⁵ is hydrogen, halogen, substituted halogen, alkyl, substituted alkyl, sulfoalkyl, alkoxy, substituted alkoxy, amino, substituted amino, aminoalkyl or substituted aminoalkyl;
R¹⁶ is hydrogen, halogen, substituted halogen, alkyl, substituted alkyl, sulfoalkyl, alkoxy, substituted alkoxy, amino, substituted amino, aminoalkyl or substituted aminoalkyl; and
R¹⁷ is alkyl, substituted alkyl, phenyl, substituted phenyl, amino alkyl, substituted aminoalkyl, sulfoalkyl, or substituted sulfoalkyl.

13. The method according to claim 13, wherein the dye has the general formula:

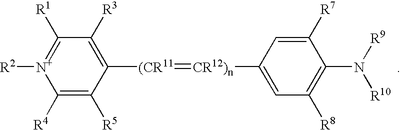

14. The method according to claim 13, wherein the dye is

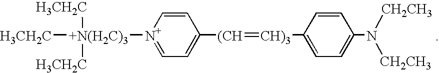

15. The method according to claim 13, wherein the dye is

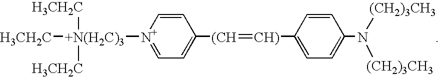

16. The method according to claim 13, wherein the dye is

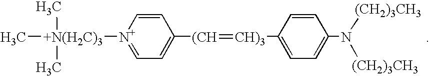

17. A method for detecting myelin in a sample, wherein the method comprises:
a. contacting the sample with a dye that selectively associates with myelin to prepare a labeling mixture, wherein the dye has the general formula 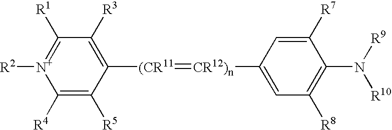
wherein
R¹ is hydrogen, halogen, substituted halogen, alkyl, substituted alkyl, sulfoalkyl, alkoxy, substituted alkoxy, amino, substituted amino, aminoalkyl or substituted aminoalkyl;
R² is alkyl, substituted alkyl, sulfoalkyl, aminoalkyl, substituted aminoalkyl, sulfoalkyl or substituted sulfoalkyl;
R³ is hydrogen, halogen, substituted halogen, alkyl, substituted alkyl, sulfoalkyl, alkoxy, substituted alkoxy, amino, substituted amino, aminoalkyl or substituted aminoalkyl;
R⁴ is hydrogen hydrogen, halogen, substituted halogen, alkyl, substituted alkyl, sulfoalkyl, alkoxy, substituted alkoxy, amino, substituted amino, aminoalkyl or substituted aminoalkyl;
R⁵ is hydrogen hydrogen, halogen, substituted halogen, alkyl, substituted alkyl, sulfoalkyl, alkoxy, substituted alkoxy, amino, substituted amino, aminoalkyl or substituted aminoalkyl;
or a member independently selected from;
R¹ in combination with R³;
R² in combination with R¹;
R² in combination with R⁴; and
R⁴ in combination with R⁵;
together with the atoms to which they are joined, form a ring which is a 5-, 6- or 7-membered cycloalkyl, a 5-, 6- or 7-membered heterocycloalkyl, a 5-, 6- or 7-membered aryl or a 5-, 6- or 7-membered heteroaryl; and
wherein R⁷ is hydrogen, halogen, substituted halogen, alkyl, substituted alkyl, sulfoalkyl, amino, substituted amino, aminoalkyl or substituted aminoalkyl;
R⁸ is hydrogen, halogen, substituted halogen, alkyl, substituted alkyl, sulfoalkyl, amino, substituted amino, aminoalkyl or substituted aminoalkyl;
R⁹ is alkyl, substituted alkyl, sulfoalkyl, aminoalkyl or substituted aminoalkyl;
R¹⁰ is alkyl, substituted alkyl, sulfoalkyl, aminoalkyl or substituted aminoalkyl; or
R⁹ and R¹⁰ in combination form a 5- or 6-membered ring, R⁹ and R⁷ in combination for a 5- or 6-membered ring or R¹⁰ and R⁸ in combination form a 5- or 6-membered ring; and
R¹¹ is hydrogen, halogen, phenyl, substituted phenyl, substituted halogen, alkyl, or substituted alkyl;
R¹² is hydrogen, halogen, phenyl, substituted phenyl, substituted halogen, alkyl, or substituted alkyl;
or R¹¹ and R¹² in combination form a 5- or 6-membered ring;
b. incubating the labeling mixture for a sufficient amount of time for the dye to associate with the myelin to form an incubated sample;
c. illuminating the incubated sample with an appropriate wavelength to form an illuminated sample; and
d. observing the illuminated sample whereby the myelin is detected.

18. The method according to claim 17, wherein the sample is a tissue section.

19. The method according to claim 18, wherein the tissues section is a brain tissue section.

20. The method according to claim 17, further comprising washing the incubated sample to remove unbound dye.

21. The method according to claim 17, further comprising contacting the sample with an additional detection reagent.

22. The method according to claim 21, wherein the additional detection reagent is an antibody, a nucleic acid stain, an ion indicator, a cytoskeleton stain, an extracellular matrix stain or an organelle stain.

23. The method according to claim 22, wherein the organelle stain is selective for mitochondria, lysozymes, nucleus, golgi, or endoplastic reticulum (ER).

24. The method according to claim 17, wherein the dye is

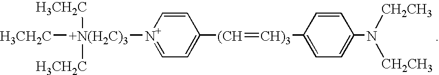

25. The method according to claim 17, wherein the dye is

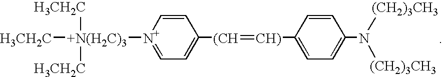

26. The method according to claim 17, wherein the dye is

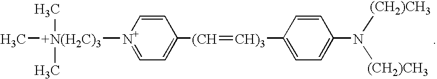

27. A kit for detecting myelin in a sample, wherein the kit comprises:
a) a lipophilic fluorescent dye that selectively associates with myelin; and,
b) instructions for using the dye to detect myelin in a sample.

28. The kit according to claim 27, further comprising an additional detection reagent.

29. The kit according to claim 28, wherein the additional detection reagent is an antibody, a nucleic acid stain, an ion indicator, a cytoskeleton stain, an extracellular matrix stain or an organelle stain.

30. The kit according to claim 29, wherein the organelle stain is selective for mitochondria, lysozymes, nucleus, golgi, or endoplastic reticulum (ER).

31. The kit according to claim 27, wherein the lipophilic dye is a merocyanine dye, a cyanine dye, a styryl dye or a carbazolylvinyl dye.

32. The kit according to claim 27, wherein the lipophilic dye has the general formula:
A-B-E
wherein A is a nitrogen heterocycle;
B is a bridge moiety; and
E is an electron pair accepting moiety that comprises either a carbonyl or nitrogen atom.

33. The kit according to claim 31, wherein the lipophilic dye is a cyanine dye.

34. The kit according to claim 31, wherein the lipophilic dye is a merocyanine dye

35. The kit according to claim 32, wherein A is 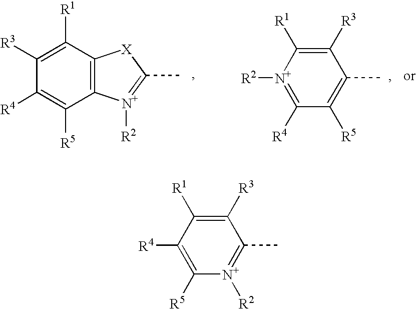
wherein,
R¹ is hydrogen, halogen, substituted halogen, alkyl, substituted alkyl, sulfoalkyl, alkoxy, substituted alkoxy, amino, substituted amino, aminoalkyl or substituted aminoalkyl;
R² is alkyl, substituted alkyl, sulfoalkyl, aminoalkyl, substituted aminoalkyl, sulfoalkyl or substituted sulfoalkyl;
R³ is hydrogen, halogen, substituted halogen, alkyl, substituted alkyl, sulfoalkyl, alkoxy, substituted alkoxy, amino, substituted amino, aminoalkyl or substituted aminoalkyl;
R⁴ is hydrogen hydrogen, halogen, substituted halogen, alkyl, substituted alkyl, sulfoalkyl, alkoxy, substituted alkoxy, amino, substituted amino, aminoalkyl or substituted aminoalkyl;
R⁵ is hydrogen hydrogen, halogen, substituted halogen, alkyl, substituted alkyl, sulfoalkyl, alkoxy, substituted alkoxy, amino, substituted amino, aminoalkyl or substituted aminoalkyl;
or a member independently selected from;
R¹ in combination with R³;
R³ in combination with R⁴; and
R⁴ in combination with R⁵;
together with the atoms to which they are joined, form a ring which is a 5-, 6- or 7-membered cycloalkyl, a 5-, 6- or 7-membered heterocycloalkyl, a 5-, 6- or 7-membered aryl or a 5-, 6- or 7-membered heteroaryl; and
X is O, S, NR⁵, or CR¹¹ R¹²wherein R¹¹ is hydrogen, halogen, phenyl, substituted phenyl, substituted halogen, alkyl, or substituted alkyl; R¹² is hydrogen, halogen, phenyl, substituted phenyl, substituted halogen, alkyl, substituted alkyl; or R¹¹ and R¹² in combination form a 5- or 6-membered ring;
B is a covalent bridge having the formula —(CR¹¹═CR¹²)ₙ—;
wherein R¹¹ is hydrogen, halogen, phenyl, substituted phenyl, substituted halogen, alkyl, or substituted alkyl;
R¹² is hydrogen, halogen, phenyl, substituted phenyl, substituted halogen, alkyl, or substituted alkyl;
or R¹¹ and R¹² in combination form a 5- or 6-membered ring;
n is 1, 2 or 3;
E is 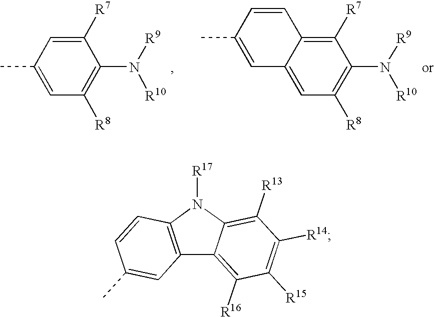
wherein R⁷ is hydrogen, halogen, substituted halogen, alkyl, substituted alkyl, sulfoalkyl, amino, substituted amino, aminoalkyl or substituted aminoalkyl;
R⁸ is hydrogen, halogen, substituted halogen, alkyl, substituted alkyl, sulfoalkyl, amino, substituted amino, aminoalkyl or substituted aminoalkyl;
R⁹ is alkyl, substituted alkyl, sulfoalkyl, aminoalkyl or substituted aminoalkyl;
R¹⁰ is alkyl, substituted alkyl, sulfoalkyl, aminoalkyl or substituted aminoalkyl; or
R⁹ and R¹⁰ in combination form a 5- or 6-membered ring, R⁹ and R⁷ in combination for a 5- or 6-membered ring or R¹⁰ and R⁸ in combination form a 5- or 6-membered ring;
R¹³ is hydrogen, halogen, substituted halogen, alkyl, substituted alkyl, sulfoalkyl, alkoxy, substituted alkoxy, amino, substituted amino, aminoalkyl or substituted aminoalkyl;
R¹⁴ is hydrogen, halogen, substituted halogen, alkyl, substituted alkyl, sulfoalkyl, alkoxy, substituted alkoxy, amino, substituted amino, aminoalkyl or substituted aminoalkyl;
R¹⁵ is hydrogen, halogen, substituted halogen, alkyl, substituted alkyl, sulfoalkyl, alkoxy, substituted alkoxy, amino, substituted amino, aminoalkyl or substituted aminoalkyl;
R¹⁶ is hydrogen, halogen, substituted halogen, alkyl, substituted alkyl, sulfoalkyl, alkoxy, substituted alkoxy, amino, substituted amino, aminoalkyl or substituted aminoalkyl; and
R¹⁷ is alkyl, substituted alkyl, phenyl, substituted phenyl, amino alkyl, substituted aminoalkyl, sulfoalkyl, or substituted sulfoalkyl.

36. The kit according to claim 35, wherein the dye has the general formula:

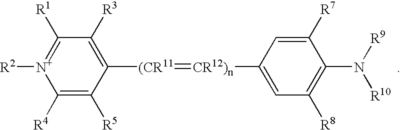

37. The kit according to claim 27, wherein the dye is

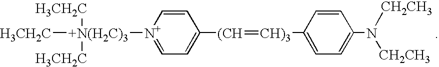

38. The kit according to claim 27, wherein the dye is

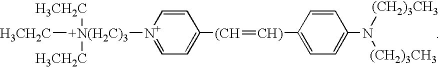

39. The kit according to claim 36, wherein the dye is [chemical structure]
